(19) United States
(12) Patent Application Publication
(10) Pub. No.: US 2003/0059806 A1
(43) Pub. Date: Mar. 27, 2003

(54) Probes for the detection of human papillomavirus

(75) Inventors: Cossette Wheeler, Placitas, NM (US); Cheri Goodall, Monmouth, OR (US)

Correspondence Address: MORGAN, LEWIS & BOCKIUS LLP, 1701 MARKET STREET, PHILADELPHIA, PA, 19103-2921, US

(73) Assignee: Science & Technology Corporation @ UNM

(21) Appl. No.: 10/162,164

(22) Filed: Jun. 3, 2002

Related U.S. Application Data
(63) Continuation of application No. PCT/US01/00389, filed on Jan. 5, 2001.

(51) Int. Cl. C12Q 1/70; C12Q 1/68; C07H 21/04

(52) U.S. Cl. 435/6; 435/5; 536/23.72; 536/24.3

(57) ABSTRACT

The present invention provides probes for HPV types 61, 62, 64, 67, 69, 70, 71, 72, CP6108, CP8304, of IS39. The present invention also provides an assay incorporating one or more of these probes. In addition, the present invention provides a method to detect one or more of HPV types 61, 62, 64, 67, 69, 70, 71, 72, CP6108, CP8304, or IS39, and subtypes and variants thereof.

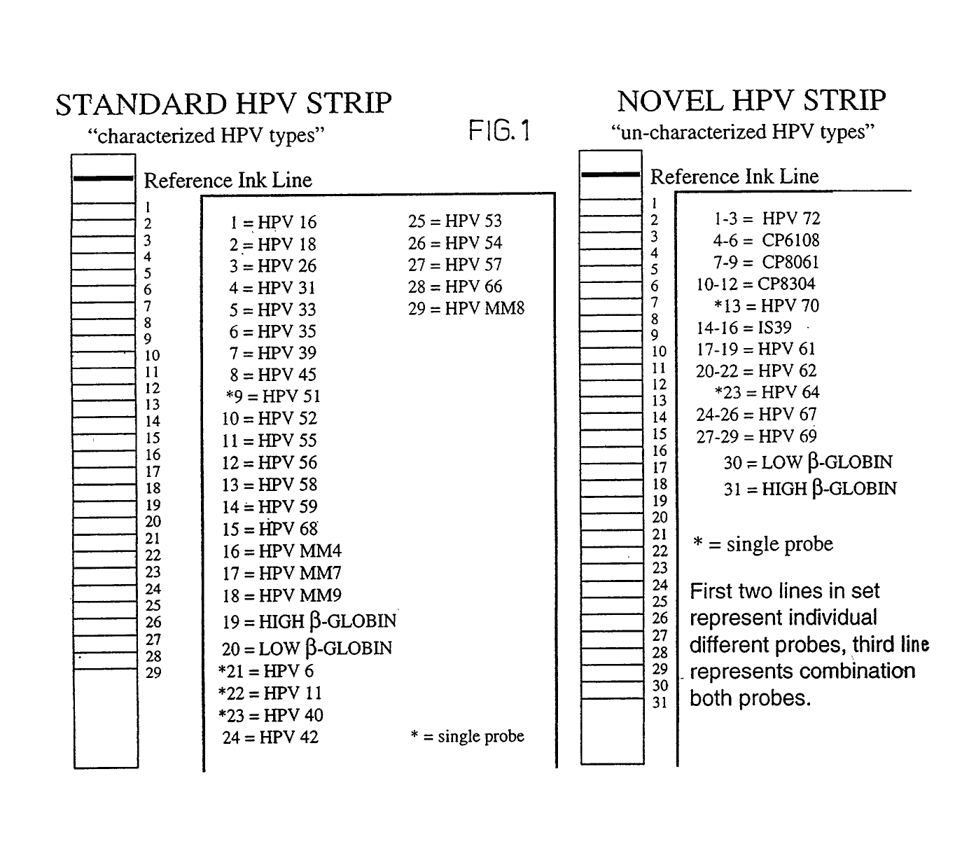

### CROSS-REFERENCE TO RELATED APPLICATIONS

This application claims priority under 35 U.S.C. §119(e) to PCT/US01/00389, filed Jan. 5, 2001.

### BACKGROUND OF THE INVENTION

Human papillomaviruses (HPVs) constitute a group of viruses associated with benign and malignant neoplasia of cutaneous and mucosal epithelia. To date, more than 100 different HPV types have been proposed. Evidence from partial sequences suggest the existence of at least 13 additional types that would qualify as novel HPVs, and it is likely that unidentified HPVs remain. About half of the reported HPVs are associated with mucosal lesions, including cervical neoplasia. A strong and consistent association has been found between infection with certain types of HPVs (e.g., HPV-16) and invasive cervical carcinoma. HPV-16 represents about 50% of the cervical cancer-associated HPV infections worldwide, although regional variations have been reported.

HPVs are characterized by a circular double-stranded DNA genome, about 8000 base pairs long, wrapped in a protein capsid 55-60 nm in diameter. The virus can be found in non-malignant lesions in the unintegrated episomal state. In these cases, disturbance of the cellular differentiation is observed and production of viral particles at late stages of the differentiation is observed. When uterine cervical carcinoma is established, a variable integration of certain sequences of viral DNA in the cellular DNA is often but not always observed.

Papillomaviruses (PVs) are defined by genomic sequence similarities rather than by classical serology. An HPV genome is defined as a new type if it is separated by a Hamming distance or dissimilarity of more than 10% in its nucleotide sequence compared with other known HPV types in the E6, E7 and L1 open reading frames (ORFs) combined. Subsequently, the taxonomy working group at the 14th International Papillomavirus Conference revised the classification criterion to the current standard which requires <90% DNA sequence homology to other HPVs in only the L1 ORF. Isolates within the same type differing by 0% to 2% in their nucleotide sequences compared with the reference sequence are referred to as variants, and those differing by 2% to 10% are referred to as subtypes.

Probes for HPVs made from DNA sequences may be obtained by various routes, particularly by genetic engineering or by manual or automatic direct synthesis. These nucleic acid sequences have the property of being matched to and of forming hybrids with complementary DNA or RNA sequences. Hybridization can be done after denaturation of target DNA in a medium to high ionic strength and at high temperature or in a basic medium. The hybrids are then detectable.

The detection of hybrids can be done by different methods. The probe may be labeled by one of the known methods for the labeling of nucleic acid probes. Known methods include radioactive labeling, for example, with phosphorus 32, and cold probes, for example, labeling with enzyme(s). In certain cases, the probe can be modified chemically to be detectable after hybridization, for example, with biotin. Alternatively, the probe itself can be used as the hybridization target for labeled fragments of complementary RNA or DNA in a reverse format.

The detection of HPV in cervical samples can be carried out by the use of molecular probes constituted from synthetic HPV-specific oligonucleotides or cloned viral DNA fragments that are labeled. Within the framework of epidemiological studies and routine diagnosis of infections by HPV, it is useful to develop a specific synthetic DNA probe labeled non-isotopically or isotopically and usable in situ or in liquid- or solid-phase assays. The specific DNA probe is able to bind to a particular portion of the HPV genome and identify cells containing a particular type of HPV.

### BRIEF SUMMARY OF THE INVENTION

The present invention provides synthetic DNA probes for HPV types 61, 62, 64, 67, 69, 70, 71, 72, CP6108, CP8304, and IS39.

In a first aspect, the present invention provides a probe which hybridizes to nucleic acid from an HPV type, the probe being selected from the group consisting of: Seq. ID No. 1 and Seq. ID No. 2, and sequences fully complementary thereto, which hybridize with HPV 72; Seq. ID No. 3, Seq. ID No. 4, Seq. ID No. 5, Seq. ID No. 6, and Seq. ID No. 7, and sequences fully complementary thereto, which hybridize with CP6108; Seq. ID No. 8, Seq. ID No. 9, Seq. ID No. 10, and Seq. ID No. 11, and sequences fully complementary thereto, which hybridize with HPV 71; Seq. ID No. 12, Seq. ID No. 13, Seq. ID No. 14, and Seq. ID No. 15, and sequences fully complementary thereto, which hybridize with CP8304; Seq. ID No. 16 and Seq. ID No. 17, and sequences fully complementary thereto, which hybridize with IS39; Seq. ID No. 18 and Seq. ID No. 19, and sequences fully complementary thereto, which hybridize with HPV 70; Seq. ID No. 20, Seq. ID No. 21, Seq. ID No. 22, and Seq. ID No. 23, and sequences fully complementary thereto, which hybridize with HPV 61; Seq. ID No. 24, Seq. ID No. 25, Seq. ID No. 26, and Seq. ID No. 27, and sequences fully complementary thereto, which hybridize with HPV 62; Seq. ID No. 28 and sequences fully complementary thereto, which hybridize with HPV 64; Seq. ID No. 29, Seq. ID No. 30, and Seq. ID No. 31, and sequences fully complementary thereto, which hybridize with HPV 67; Seq. ID No. 32, Seq. ID No. 33, Seq. ID No. 34, and Seq. ID No. 35, and sequences fully complementary thereto, which hybridize with HPV 69.

In a second aspect, the present invention provides an assay for determining the presence of at least one HPV type, the assay comprising: a solid support; and at least one probe deposited on the solid support, the at least one probe being able to hybridize to nucleic acid from an HPV type, and the at least one probe being selected from the group consisting of: Seq. ID No. 1 and Seq. ID No. 2, and sequences fully complementary thereto, which hybridize with HPV 72; Seq. ID No. 3, Seq. ID No. 4, Seq. ID No. 5, Seq. ID No. 6, and Seq. ID No. 7, and sequences fully complementary thereto, which hybridize with CP6108; Seq. ID No. 8, Seq. ID No. 9, Seq. ID No. 10, and Seq. ID No. 11, and sequences fully complementary thereto, which hybridize with HPV 71; Seq. ID No. 12, Seq. ID No. 13, Seq. ID No. 14, and Seq. ID No. 15, and sequences fully complementary thereto, which hybridize with CP8304; Seq. ID No. 16 and Seq. ID No. 17, and sequences fully complementary thereto, which hybridize with IS39; Seq. ID No. 18 and Seq. ID No. 19, and sequences fully complementary thereto, which hybridize with HPV 70; Seq. ID No. 20, Seq. ID No. 21, Seq. ID No. 22, and Seq. ID No. 23, and sequences fully complementary thereto, which hybridize with HPV 61; Seq. ID No. 24, Seq. ID No. 25, Seq. ID No. 26, and Seq. ID No. 27, and sequences fully complementary thereto, which hybridize with HPV 62; Seq. ID No. 28 and sequences fully complementary thereto, which hybridize with HPV 64; Seq. ID No. 29, Seq. ID No. 30, and Seq. ID No. 31, and sequences fully complementary thereto, which hybridize with HPV 67; Seq. ID No. 32, Seq. ID No. 33, Seq. ID No. 34, and Seq. ID No. 35, and sequences fully complementary thereto, which hybridize with HPV 69.

In a third aspect, the present invention provides a method for determining the presence of at least one HPV type in a sample obtained from a human comprising the steps of: a) adding a sample obtained from a human to an assay; the assay including a solid support and at least one probe deposited thereon; b) incubating the sample and assay combination for a period of time sufficient to permit binding of any human papillomavirus nucleic acid portions of the sample to the at least one probe on the assay; and c) observing the location of any binding of HPV types contained in the sample to the probes to determine the presence or absence of HPV 61, HPV 62, HPV 64, HPV 67, HPV 69, HPV 70, HPV 71, HPV 72, CP6108, CP8304, or IS39, wherein the at least one probe is selected from the group consisting of: Seq. ID No. 1 and Seq. ID NO. 2, and sequences fully complementary thereto, which hybridize with HPV 72; Seq. ID No. 3, Seq. ID No. 4, Seq. ID No. 5, Seq. ID No. 6, and Seq. ID No. 7, and sequences fully complementary thereto, which hybridize with CP6108; Seq. ID No. 8, Seq. ID No. 9, Seq. ID No. 10, and Seq. ID No. 11, and sequences fully complementary thereto, which hybridize with HPV 71; Seq. ID No. 12, Seq. ID No. 13, Seq. ID No. 14, and Seq. ID No. 15, and sequences fully complementary thereto, which hybridize with CP8304; Seq. ID No. 16 and Seq. ID No. 17, and sequences fully complementary thereto, which hybridize with IS39; Seq. ID No. 18 and Seq. ID No. 19, and sequences fully complementary thereto, which hybridize with HPV 70; Seq. ID No. 20, Seq. ID No. 21, Seq. ID No. 22, and Seq. ID No. 23, and sequences fully complementary thereto, which hybridize with HPV 61; Seq. ID No. 24, Seq. ID No. 25, Seq. ID No. 26, and Seq. ID No. 27, and sequences fully complementary thereto, which hybridize with HPV 62; Seq. ID No. 28 and sequences fully complementary thereto, which hybridize with HPV 64; Seq. ID No. 29, Seq. ID No. 30, and Seq. ID No. 31, and sequences fully complementary thereto, which hybridize with HPV 67; Seq. ID No. 32, Seq. ID No. 33, Seq. ID No. 34, and Seq. ID No. 35, and sequences fully complementary thereto, which hybridize with HPV 69.

In a fourth aspect, the present invention provides a probe which hybridizes to nucleic acid from an HPV type, the probe is selected from the group consisting of all of the majority sequences of the group of oligonucleotide sequences consisting of: Seq. ID No. 2 and sequences fully complementary thereto, which hybridize with HPV 72; Seq. ID No. 3, Seq. ID No. 4, Seq. ID No. 5, Seq. ID No. 6, and Seq. ID No. 7, and sequences fully complementary thereto, which hybridize with CP6108; Seq. ID No. 8, Seq. ID No. 9, Seq. ID No. 10, and Seq. ID No. 11, and sequences fully complementary thereto, which hybridize with HPV 71; Seq. ID No. 12, Seq. ID No. 13, Seq. ID No. 14, and Seq. ID No. 15, and sequences fully complementary thereto, which hybridize with CP8304; Seq. ID No. 16 and Seq. ID No. 17, and sequences fully complementary thereto, which hybridize with IS39; Seq. ID No. 18 and Seq. ID No. 19, and sequences fully complementary thereto, which hybridize with HPV 70; Seq. ID No. 20, Seq. ID No. 21, Seq. ID No. 22, and Seq. ID No. 23, and sequences fully complementary thereto, which hybridize with HPV 61; Seq. ID No. 24, Seq. ID No. 25, Seq. ID No. 26, and Seq. ID No. 27, and sequences fully complementary thereto, which hybridize with HPV 62; Seq. ID No. 28 and sequences fully complementary thereto, which hybridize with HPV 64; Seq. ID No. 29, Seq. ID No. 30, and Seq. ID No. 31, and sequences fully complementary thereto, which hybridize with HPV 67; Seq. ID No. 33, Seq. ID No. 34, and Seq. ID No. 35, and sequences fully complementary thereto, which hybridize with HPV 69.

### BRIEF DESCRIPTION OF THE DRAWINGS

The invention will be described in conjunction with the accompanying drawing, in which:

The sole FIGURE is a schematic illustration of an assay of the present invention with a description of the probes associated with each line of the assay.

### DETAILED DESCRIPTION OF THE PREFERRED EMBODIMENT

A Sequence Listing accompanies this application and is incorporated herein in its entirety.

Definitions

To aid in understanding the invention, several terms are defined below:

Unless specifically indicated otherwise, for the purposes of the present invention, the nucleotides T (thymine) and U (uracil) are interchangeable in the oligonucleotide sequences of the present invention.

For the purposes of the present invention, the terms “nucleic acid” and “oligonucleotide” refer to: primers, probes and oligomer fragments. The terms are generic to: polydeoxyribonucleotides (containing 2-deoxy-D-ribose), to polyribonucleotides (containing D-ribose), and to any other type of polynucleotide which is an N glycoside of a purine or pyrimidine base, or modified purine or pyrimidine base. There is no intended distinction in length between the terms “nucleic acid” and “oligonucleotide,” and these terms are used interchangeably for the purposes of the present invention. These terms refer only to the primary structure of the molecule. Thus, these terms include: double- and single-stranded DNA, as well as double- and single-stranded RNA. The exact size of an oligonucleotide depends on many factors and the ultimate function or use of the oligonucleotide. Oligonucleotides can be prepared by any suitable method, including, for example, cloning and restriction of appropriate sequences and direct chemical synthesis by a method such as the phosphotriester method of Narang et al., 1979, Meth. Enzymol. 68:90-99; the phosphodiester method of Brown et al., 1979, Meth. Enzymol. 68:109-151; the diethylphosphoramidite method of Beaucage et al., 1981, Tetrahedron Lett. 22:1859-1862; and the solid support method of U.S. Pat. No. 4,458,066 to Caruthers et al., each incorporated herein by reference, or suitable combination of the above. A review of synthesis methods is provided in Goodchild, 1990, Bioconjugate Chemistry 1(3):165-187, incorporated herein by reference.

For the purposes of the present invention, the term “target sequence” refers to a region of DNA or RNA which is to be analyzed.

For the purposes of the present invention, the term “hybridization” refers to the formation of a duplex structure by two single-stranded nucleic acids due to complementary base pairing. Hybridization can occur between complementary nucleic acid strands or between nucleic acid strands that contain minor regions of mismatch. Conditions under which only complementary nucleic acid strands will hybridize are referred to as “stringent hybridization conditions.” Two single-stranded nucleic acids that are complementary except for minor regions of mismatch are referred to as “substantially complementary.” Stable duplexes of substantially complementary sequences can be achieved under less stringent hybridization conditions. Those skilled in the art of nucleic acid technology can determine duplex stability by empirically considering a number of variables, including, for example, the length and base pair composition of the oligonucleotides, ionic strength, temperature, and incidence of mismatched base pairs.

For the purposes of the present invention, the term “probe” refers to an oligonucleotide which forms a duplex structure with a target RNA or DNA sequence to be analyzed due to complementary base pairing. A probe of the present invention generally includes a “hybridizing region,” a region of the oligonucleotide probe corresponding to a region of the target sequence. For the purposes of the present invention, the term “corresponding” means identical to or complementary to the designated nucleic acid. An oligonucleotide probe can either consist entirely of the hybridizing region or can contain additional features which allow for the detection or immobilization of the probe, but do not alter the hybridization characteristics of the hybridizing region. As used herein, the term “probe” also refers to a set of oligonucleotides wherein the oligonucleotides of the set provide sufficient sequence variance of a target hybridization region to enable hybridization with each respective member of a given set of target sequence variants. For example, because within each HPV type there are subtypes and variants which contain minor sequence variation of different degrees, a type-specific probe of the present invention may contain a set of oligonucleotides for hybridization with each of the subtype and/or variant sequences. Additionally, a probe of the present invention may consist of one or more oligonucleotides which contain mismatches with some or all members of a given set of target sequence subtypes or variants, but contain sufficient regions of complementarity with each target sequence variant so as to enable hybridization with all target sequence variants under suitable conditions.

For the purposes of the present invention, the terms “sequence-specific oligonucleotide” and “SSO” refer to oligonucleotide probes wherein the hybridization region is exactly complementary to the sequence to be detected. The use of the term “stringent hybridization conditions” refers to conditions under which the probe will hybridize only to that exactly complementary target sequence, and which allow the detection of the specific target sequence. Stringent hybridization conditions, sometimes referred to as “stringent conditions,” are well known in the art (see, e.g., Sambrook et al., 1985, Molecular Cloning—A Laboratory Manual, Cold Spring Harbor Laboratory, Cold Spring Harbor, N.Y., incorporated herein by reference). Stringent hybridization conditions are sequence dependent and will be different in different circumstances. Generally, stringent hybridization conditions are selected to be about 5° C. lower than the thermal melting point (Tₘ) for the specific sequence at a defined ionic strength and pH. The Tₘ is the temperature (under defined ionic strength and pH) at which 50% of the base pairs have dissociated. Typically, stringent hybridization conditions will be those in which the salt concentration is at least about 0.2 molar at pH 7 and the temperature is typically greater than 50° C.; however, this is sequence dependent. Relaxing the stringency of the hybridizing conditions will allow sequence mismatches to be formed and tolerated. The degree of mismatch tolerated can be controlled by suitable adjustment of the hybridization conditions, including salt concentration and pH level.

For the purposes of the present invention, the term “majority sequence” of an oligonucleotide sequence refers to a nucleic acid sequence containing at least 50% of the base pairs, in sequence order, of the oligonucleotide. For example, with respect to Seq. ID No. 2 below: cgtgagtatc ttcgccacac, one majority sequence is cgtgagtatc tt. A second majority sequence of Seq. ID No. 2 is gtatc ttcgc. A third majority sequence would be guauc uucgc. For the purposes of the present invention, a majority sequence may be an “interior majority sequence” or a “mixed majority sequence.” For the purposes of the present invention, an “interior majority sequence” is entirely contained within the sequence of the subject nucleotide. For example, the sequences cgtgagtatc tt, gtatc ttcgc, and guauc uucgc are interior sequences of the oligonucleotide sequence of Seq. ID No. 2. For the purposes of the present invention, a “mixed majority sequence” includes a “matching region” which contains at least 50% of the base pairs of a given oligonucleotide and at least one “non-matching region” which does not match the base pairs of the oligonucleotide. For example, aaaaaaaaaaacgtgagtatc tt is a mixed majority sequence of Seq. ID No. 2. The sequence acacacacac cgtgagtatc ttgtgtgtgt is also a mixed majority sequence of Seq. ID No. 2. Another mixed majority sequence of Seq. ID No. 2 is cucucguauc uucgcugugu. As can be seen, a given oligonucleotide sequence can have many majority sequences.

Description

Within the past several years, several human papillomavirus (HPV) genomes and L1 fragments have been cloned, including HPV types 61, 62, 64, 67, 69, 70, 71, 72, CP6108, CP8304, and IS39. HPV types 61, 62, 71, 72, CP6108, and CP8304 represent a discrete phylogenetic cluster.

One or more type-specific oligonucleotide probes of the present invention for HPV types 61, 62, 64, 67, 69, 70, 71, 72, CP6108, CP8304, and IS39 are preferably designed by a sequence alignment program such as the PileUp™ Program (Genetics Computer Group, Madison, Wis.) to identify potential probes using the following criteria: length of 18-22 base pairs (bp); G/C to A/T ratio approximately 50:50; no strings of A, T, or A and T longer than four; Tₘ of 58°-62°; including GC clamps if possible on ends; gross visual uniqueness of sequence. A sequence is “grossly visually unique” for the purposes of the present invention, if it contains at least 4 mismatches when compared in an aligned region to all other HPV types with a preference for probes with greater than 4 mismatches.

Suitable solid-support for the assays of the present invention include nylon and mitrocellulose membrane genotyping strips, and other solid-phases, including but not limited to glass or plastic. The oligonucleotide probes of the present invention can be deposited on the solid support to form an assay by using various techniques, such as an automated system, a dot or line-blot manifold, etc.

Samples for testing for the presence of HPVs using the method of the present invention may be prepared by collecting cervical samples that have been collected in a specimen transport medium such as STM™ (Digene Corp., Silver Spring, Md.) or PreservCyt™ (Cytyc, Boxborough, Mass.), or by collecting in any suitable buffer such as phosphate buffered saline (PBS), Tris, etc., or cell or viral culture medium, or tissues that have been derived from archival paraffin-embedded materials, and digesting the specimens with a suitable proteinase, such as proteinase K and a detergent such as Laureth-12, NP-40, Tween™, etc. Then the digested material or portion thereof is mixed with a suitable precipitation solution, such as ethanol containing 10% ammonium acetate and the nucleic acids (NAs) present in the precipitation solution are allowed to precipitate overnight at −20° C. The precipitated NAs are then centrifuged to create a pellet, after which the supernatant is discarded, and a residual crude NA pellet is optionally washed with a suitable material such as ethanol, then is allowed to dry, preferably at about room temperature or slightly warmer to increase the evaporation rate. The pellet is then preferably, resuspended in a suitable suspension medium, such as Tris-EDTA, and incubated for a suitable time at a temperature sufficient to inactivate the proteinase, preferably about 95° C. Prior to amplification, the crude nucleotide digest is, preferably, allowed to reach room temperature and is further centrifuged to minimize aeresolization. A portion of the digest is amplified using a suitable PCR amplification technique, such as by using a biotinylated version of the MY09-MY11-HMB01 L1 consensus primer system and AmpliTaq Gold™ DNA polymerase (Perkin Elmer, Foster City, Calif.).

### EXAMPLE

Specimen Processing

Cervical specimens were collected in 1.0 ml of specimen transport medium, and processed by adding 30 μl of digestion solution to achieve a final concentration of 200 μg of proteinase K per ml and 0.1% Laureth-12. Digestion was conducted at 56° C. for 1 hour. A 300 μl aliquot of the digested material was added to 1.0 ml of absolute ethanol containing 10% ammonium acetate and precipitated overnight at −20° C. The precipitated DNA was centrifuged for 30 minutes at 13,000×g at 4° C. The supernatant was immediately discarded and the crude DNA pellet was dried overnight at room temperature. The pellet was resuspended in 150 μl of TE (20 mM Tris, 1 mM EDTA) and incubated for 15 minutes at 95° C. to inactivate the proteinase K. Prior to amplification, the crude digests were allowed to reach room temperature and were centrifuged briefly. Six μl of each sample was amplified with 5′-biotinylated MY09/11 (50 pmol), HMB01, GH20, PC04 (5 pmol each), in the presence of 1×PCR Buffer II, 6 mM MgCl₂, 200 μmol each of dATP, dCTP, and dGTP, 600 μmol dUTP, and 7.5 units AmpliTaq Gold™ DNA polymerase (Perkin Elmer, Foster City, Calif.). Amplifications were performed in a Perkin-Elmer TC9600 thermal cycler using the ultrasensitive profile of a 9-minute Amplitaq Gold™ activation at 95° C., followed by 95° C. for 1 minute, 55° C. for 1 minute, and 72° C. for 1 minute, for a total of 40 cycles. This was followed by a final extension at 72° C. for 5 minutes and amplification reactions were stored at −20° C. A similar procedure for specimen processing which is suitable for the purposes of the present invention is described in Gravitt et al., “Genotyping of 27 Human Papillomavirus Types by Using L1 Consensus PCR Products by a Single-Hybridization, Reverse Line Blot Detection Method” in J. Clin. Micro. (October 1998), and Manos et al., 1989, Cancer Cells 7:209-214, the entire contents and disclosures of which are hereby incorporated by reference.

Probe Development

Type-specific oligonucleotide probes for HPV types 61, 62, 64, 67, 69, 70, 71, 72, CP6108, CP8304, and IS39 were designed by using the PileUp Program™ (GCG) to identify potential probes using the following criteria: length of 18-22 base pairs (bp); G/C to A/T ratio approximately 50:50; no strings of A, T, or A and T longer than four; Tₘ of 58°-62°; including GC clamps if possible on ends; and gross visual uniqueness of sequence.

Probes which were determined to have at least four mismatches when compared to all known HPV types and subtypes or variants using FASTA (GCG) were tested on nylon membrane genotyping strips. When possible, the two best performing probes in terms of specificity and sensitivity were chosen for the final genotyping strip. In a few cases, only one probe was selected.

HPV Genotyping

An HPV genotyping strip was formed as shown in the drawing FIGURE. The strip was set up to detect 11 individual genotypes and human betaglobin DNA. Two bovine serum albumin (BSA)-conjugated probes per HPV type, each corresponding to a hypervariable region within the MY09-MY11 amplicon, were deposited each as a separate line and also together as a single line for each of the following HPV types: 61, 62, 67, 69, 71, 72, CP6108, CP8304 and IS39. In the case of HPV types 64 and 70, and the β-globin probe, only a single probe was deposited. The hybridization procedure used has been previously described in Gravitt, et al., “Genotyping of 27 Human Papillomavirus Types by Using L1 Consensus PCR Products by a Single-Hybridization, Reverse Line Blot Detection Method” in J. Clin. Micro. (October 1998), previously cited and incorporated by reference.

Although the present invention has been fully described in conjunction with the preferred embodiment thereof with reference to the accompanying drawings, it is to be understood that various changes and modifications may be apparent to those skilled in the art. Such changes and modifications are to be understood as included within the scope of the present invention as defined by the appended claims, unless they depart therefrom.

What is claimed is:

1. A probe which hybridizes to nucleic acid from an HPV type, said probe being selected from the group consisting of:
Seq. ID No. 1 and Seq. ID No. 2, and sequences fully complementary thereto, which hybridize with HPV 72;
Seq. ID No. 3, Seq. ID No. 4, Seq. ID No. 5, Seq. ID No. 6, and Seq. ID No. 7, and sequences fully complementary thereto, which hybridize with CP6108;
Seq. ID No. 8, Seq. ID No. 9, Seq. ID No. 10, and Seq. ID No. 11, and sequences fully complementary thereto, which hybridize with HPV 71;
Seq. ID No. 12, Seq. ID No. 13, Seq. ID No. 14, and Seq. ID No. 15, and sequences fully complementary thereto, which hybridize with CP8304;
Seq. ID No. 16 and Seq. ID No. 17, and sequences fully complementary thereto, which hybridize with IS39;
Seq. ID No. 18 and Seq. ID No. 19, and sequences fully complementary thereto, which hybridize with HPV 70;
Seq. ID No. 20, Seq. ID No. 21, Seq. ID No. 22, and Seq. ID No. 23, and sequences fully complementary thereto, which hybridize with HPV 61;
Seq. ID No. 24, Seq. ID No. 25, Seq. ID No. 26, and Seq. ID No. 27, and sequences fully complementary thereto, which hybridize with HPV 62;
Seq. ID No. 28 and sequences fully complementary thereto, which hybridize with HPV 64;
Seq. ID No. 29, Seq. ID No. 30, and Seq. ID No. 31, and sequences fully complementary thereto, which hybridize with HPV 67;
Seq. ID No. 32, Seq. ID No. 33, Seq. ID No. 34, and Seq. ID No. 35, and sequences fully complementary thereto, which hybridize with HPV 69.

2. The probe of claim 1, wherein said HPV type is HPV 72, and wherein said probe is Seq. ID No. 1, and sequences fully complementary thereto.

3. The probe of claim 1, wherein said HPV type is HPV 72, and wherein said probe is Seq. ID No. 2, and sequences fully complementary thereto.

4. The probe of claim 1, wherein said HPV type is CP6108, and wherein said probe is selected from the group of probes consisting of: Seq. ID No. 3, Seq. ID No. 4, Seq. ID No. 5, Seq. ID No. 6, and Seq. ID No. 7, and sequences fully complementary thereto.

5. The probe of claim 1, wherein said HPV type is HPV 71, and wherein said probe is selected from the group of probes consisting of: Seq. ID No. 8, Seq. ID No. 9, Seq. ID No. 10, and Seq. ID No. 11, and sequences fully complementary thereto.

6. The probe of claim 1, wherein said HPV type is CP8304, and wherein said probe is selected from the group of probes consisting of: Seq. ID No. 12, Seq. ID No. 13, Seq. ID No. 14, and Seq. ID No. 15, and sequences fully complementary thereto.

7. The probe of claim 1, wherein said HPV type is IS39, and wherein said probe is selected from the group of probes consisting of: Seq. ID No. 16 and Seq. ID No. 17, and sequences fully complementary thereto.

8. The probe of claim 1, wherein said HPV type is HPV 70, and wherein said probe is selected from the group of probes consisting of: Seq. ID No. 18 and Seq. ID No. 19, and sequences fully complementary thereto.

9. The probe of claim 1, wherein said HPV type is HPV 61, and wherein said probe is selected from the group of probes consisting of: Seq. ID No. 20, Seq. ID No. 21, Seq. ID No. 22, and Seq. ID No. 23, and sequences fully complementary thereto.

10. The probe of claim 1, wherein said HPV type is HPV 62, and wherein said probe is selected from the group of probes consisting of: Seq. ID No. 24, Seq. ID No. 25, Seq. ID No. 26, and Seq. ID No. 27, and sequences fully complementary thereto.

11. The probe of claim 1, wherein said HPV type is HPV 64, and wherein said probe is Seq. ID No. 28, and sequences fully complementary thereto.

12. The probe of claim 1, wherein said HPV type is HPV 67, and wherein said probe is selected from the group of probes consisting of: Seq. ID No. 29, Seq. ID No. 30, and Seq. ID No. 31, and sequences fully complementary thereto.

13. The probe of claim 1, wherein said HPV type is HPV 69, and wherein said probe is Seq. ID No. 32, and sequences fully complementary thereto.

14. The probe of claim 1, wherein said HPV type is HPV 69, and wherein said probe is selected from the group of probes consisting of: Seq. ID No. 33, Seq. ID No. 34, and Seq. ID No. 35, and sequences fully complementary thereto.

15. An assay for determining the presence of at least one HPV type, said assay comprising:
a solid support; and
at least one probe deposited on said solid support, said at least one probe being able to hybridize to nucleic acid from an HPV type, and said at least one probe being selected from the group consisting of:
Seq. ID No. 1 and Seq. ID No. 2, and sequences fully complementary thereto, which hybridize with HPV 72;
Seq. ID No. 3, Seq. ID No. 4, Seq. ID No. 5, Seq. ID No. 6, and Seq. ID No. 7, and sequences fully complementary thereto, which hybridize with CP6108;
Seq. ID No. 8, Seq. ID No. 9, Seq. ID No. 10, and Seq. ID No. 11, and sequences fully complementary thereto, which hybridize with HPV 71;
Seq. ID No. 12, Seq. ID No. 13, Seq. ID No. 14, and Seq. ID No. 15, and sequences fully complementary thereto, which hybridize with CP8304;
Seq. ID No. 16 and Seq. ID No. 17, and sequences fully complementary thereto, which hybridize with IS39;
Seq. ID No. 18 and Seq. ID No. 19, and sequences fully complementary thereto, which hybridize with HPV 70;
Seq. ID No. 20, Seq. ID No. 21, Seq. ID No. 22, and Seq. ID No. 23, and sequences fully complementary thereto, which hybridize with HPV 61;
Seq. ID No. 24, Seq. ID No. 25, Seq. ID No. 26, and Seq. ID No. 27, and sequences fully complementary thereto, which hybridize with HPV 62;
Seq. ID No. 28, and sequences fully complementary thereto, which hybridize with HPV 64;
Seq. ID No. 29, Seq. ID No. 30, and Seq. ID No. 31, and sequences fully complementary thereto, which hybridize with HPV 67;
Seq. ID No. 32, Seq. ID No. 33, Seq. ID No. 34, and Seq. ID No. 35, and sequences fully complementary thereto, which hybridize with HPV 69.

16. The assay of claim 15, wherein said at least one probe comprises at least two different probes.

17. The assay of claim 15, wherein said HPV type is HPV 72, and wherein said at least one probe is Seq. ID No. 1, and sequences fully complementary thereto.

18. The assay of claim 15, wherein said HPV type is HPV 72, and wherein said at least one probe is Seq. ID No. 2, and sequences fully complementary thereto.

19. The assay of claim 15, wherein said HPV type is CP6108, and wherein said at least one probe is selected from the group of probes consisting of: Seq. ID No. 3, Seq. ID No. 4, Seq. ID No. 5, Seq. ID No. 6, and Seq. ID No. 7, and sequences fully complementary thereto.

20. The assay of claim 15, wherein said HPV type is HPV 71, and wherein said at least one probe is selected from the group of probes consisting of: Seq. ID No. 8, Seq. ID No. 9, Seq. ID No. 10, and Seq. ID No. 1, and sequences fully complementary thereto.

21. The assay of claim 15, wherein said HPV type is CP8304, and wherein said at least one probe is selected from the group of probes consisting of: Seq. ID No. 12, Seq. ID No. 13, Seq. ID No. 14, and Seq. ID No. 15, and sequences fully complementary thereto.

22. The assay of claim 15, wherein said HPV type is IS39, and wherein said at least one probe is selected from the group of probes consisting of: Seq. ID No. 16 and Seq. ID No. 17, and sequences fully complementary thereto.

23. The assay of claim 15, wherein said HPV type is HPV 70, and wherein said at least one probe is selected from the group of probes consisting of: Seq. ID No. 18 and Seq. ID No. 19, and sequences fully complementary thereto.

24. The assay of claim 15, wherein said HPV type is HPV 61, and wherein said at least one probe is selected from the group of probes consisting of: Seq. ID No. 20, Seq. ID No. 21, Seq. ID No. 22, and Seq. ID No. 23, and sequences fully complementary thereto.

25. The assay of claim 15, wherein said HPV type is HPV 62, and wherein said at least one probe is selected from the group of probes consisting of: Seq. ID No. 24, Seq. ID No. 25, Seq. ID No. 26, and Seq. ID No. 27, and sequences fully complementary thereto.

26. The assay of claim 15, wherein said HPV type is HPV 64, and wherein said at least one probe is Seq. ID No. 28, and sequences fully complementary thereto.

27. The assay of claim 15, wherein said HPV type is HPV 67, and wherein said at least one probe is selected from the group of probes consisting of: Seq. ID No. 29, Seq. ID No. 30, and Seq. ID No. 31, and sequences fully complementary thereto.

28. The assay of claim 15, wherein said HPV type is HPV 69, and wherein said at least one probe is Seq. ID No. 32, and sequences fully complementary thereto.

29. The assay of claim 15, wherein said HPV type is HPV 69, and wherein said at least one probe is selected from the group of probes consisting of: Seq. ID No. 33, Seq. ID No. 34, and Seq. ID No. 35, and sequences fully complementary thereto.

30. A method for determining the presence of at least one HPV type in a sample obtained from a human comprising the steps of:
a) adding a sample obtained from a human to an assay, the assay including a solid support and at least one probe deposited thereon;
b) incubating the sample and assay combination for a period of time sufficient to permit binding of any human papillomavirus nucleic acid portions of the sample to the at least one probe on the assay; and
c) observing the location of any binding of HPV types contained in the sample to the probes to determine the presence or absence of HPV-6 1, HPV-62, HPV-64, HPV-67, HPV-69, HPV-70, HPV-71, HPV-72, CP6108, CP8304, or IS39, wherein said at least one probe is selected from the group consisting of:
Seq. ID No. 1 and Seq. ID No. 2, and sequences fully complementary thereto, which hybridize with HPV 72;
Seq. ID No. 3, Seq. ID No. 4, Seq. ID No. 5, Seq. ID No. 6, and Seq. ID No. 7, and sequences fully complementary thereto, which hybridize with CP6108;
Seq. ID No. 8, Seq. ID No. 9, Seq. ID No. 10, and Seq. ID No. 11, and sequences fully complementary thereto, which hybridize with HPV 71;
Seq. ID No. 12, Seq. ID No. 13, Seq. ID No. 14, and Seq. ID No. 15, and sequences fully complementary thereto, which hybridize with CP8304;
Seq. ID No. 16 and Seq. ID No. 17, and sequences fully complementary thereto, which hybridize with IS39;
Seq. ID No. 18 and Seq. ID No. 19, and sequences fully complementary thereto, which hybridize with HPV 70;
Seq. ID No. 20, Seq. ID No. 21, Seq. ID No. 22, and Seq. ID No. 23, and sequences fully complementary thereto, which hybridize with HPV 61;
Seq. ID No. 24, Seq. ID No. 25, Seq. ID No. 26, and Seq. ID No. 27, and sequences fully complementary thereto, which hybridize with HPV 62;
Seq. ID No. 28 and sequences fully complementary thereto, which hybridize with HPV 64;
Seq. ID No. 29, Seq. ID No. 30, and Seq. ID No. 32, and sequences fully complementary thereto, which hybridize with HPV 67;
Seq. ID No. 32, Seq. ID No. 33, Seq. ID No. 34, and Seq. ID No. 35, and sequences fully complementary thereto, which hybridize with HPV 69.

31. The method of claim 30, wherein said at least one probe comprises at least two different probes.

32. A probe which hybridizes to nucleic acid from an HPV type, said probe selected from the group consisting of all of the majority sequences of the group of oligonucleotide sequences consisting of:
Seq. ID No. 2, and sequences fully complementary thereto, which hybridize with HPV 72;
Seq. ID No. 3, Seq. ID No. 4, Seq. ID No. 5, Seq. ID No. 6, and Seq. ID No. 7, and sequences fully complementary thereto, which hybridize with CP6108;
Seq. ID No. 8, Seq. ID No. 9, Seq. ID No. 10, and Seq. ID No. 11, and sequences fully complementary thereto, which hybridize with HPV 71;
Seq. ID No. 12, Seq. ID No. 13, Seq. ID No. 14, and Seq. ID No. 15, and sequences fully complementary thereto, which hybridize with CP8304;
Seq. ID No. 16 and Seq. ID No. 17, and sequences fully complementary thereto, which hybridize with IS39;
Seq. ID No. 18 and Seq. ID No. 19, and sequences fully complementary thereto, which hybridize with HPV 70;
Seq. ID No. 20, Seq. ID No. 21, Seq. ID No. 22, and Seq. ID No. 23, and sequences fully complementary thereto, which hybridize with HPV 61;
Seq. ID No. 24, Seq. ID No. 25, Seq. ID No. 26, and Seq. ID No. 27, and sequences fully complementary thereto, which hybridize with HPV 62;
Seq. ID No. 28 and sequences fully complementary thereto, which hybridize with HPV 64;
Seq. ID No. 29, Seq. ID No. 30, and Seq. ID No. 31, and sequences fully complementary thereto, which hybridize with HPV 67;
Seq. ID No. 33, Seq. ID No. 34, and Seq. ID No. 35, and sequences fully complementary thereto, which hybridize with HPV 69.

33. The probe of claim 32, wherein said probe is an interior majority sequence of one of said oligonucleotide sequences.

34. The probe of claim 32, wherein said probe is a mixed majority sequence of one of said oligonucleotide sequences.


SEQUENCE LISTING (35 sequences)

SEQ ID NO: 1 - DNA, 19 bp, Papillomavirus sylvilagi
gccacagcgtcctctgtat

SEQ ID NO: 2 - DNA, 20 bp, Papillomavirus sylvilagi
cgtgagtatcttcgccacac

SEQ ID NO: 3 - DNA, 19 bp, Papillomavirus sylvilagi
tgctgcttcccagtctgcc

SEQ ID NO: 4 - DNA, 22 bp, Papillomavirus sylvilagi
ccacagaatacagttctacacg

SEQ ID NO: 5 - DNA, 21 bp, Papillomavirus sylvilagi
ctatcgctttcttacctctcg

SEQ ID NO: 6 - DNA, 19 bp, Papillomavirus sylvilagi
gcactgctgccccagaacc

SEQ ID NO: 7 - DNA, 22 bp, Papillomavirus sylvilagi
cgtttgtccactgatcttgatc

SEQ ID NO: 8 - DNA, 21 bp, Papillomavirus sylvilagi
gtccatctgtgctaccaaaac

SEQ ID NO: 9 - DNA, 21 bp, Papillomavirus sylvilagi
ctacttacatcgcatggatgc

SEQ ID NO: 10 - DNA, 20 bp, Papillomavirus sylvilagi
gttcttaccacctcctactg

SEQ ID NO: 11 - DNA, 21 bp, Papillomavirus sylvilagi
gcagatcttacattttgggag

SEQ ID NO: 12 - DNA, 19 bp, Papillomavirus sylvilagi
gcacagctacatctgctgc

SEQ ID NO: 13 - DNA, 20 bp, Papillomavirus sylvilagi
gctgcagaatacaaggcctc

SEQ ID NO: 14 - DNA, 20 bp, Papillomavirus sylvilagi
cccaaagaggacccttatgc

SEQ ID NO: 15 - DNA, 21 bp, Papillomavirus sylvilagi
gccgacatgtcattttggaca

SEQ ID NO: 16 - DNA, 20 bp, Papillomavirus sylvilagi
gcagcaacctcttgtcaacg

SEQ ID NO: 17 - DNA, 20 bp, Papillomavirus sylvilagi
gcacagacattcactccaac

SEQ ID NO: 18 - DNA, 18 bp, Papillomavirus sylvilagi
gcctgcaccgaaacggcc

SEQ ID NO: 19 - DNA, 22 bp, Papillomavirus sylvilagi
cgtataggtatttacaatcagc

SEQ ID NO: 20 - DNA, 18 bp, Papillomavirus sylvilagi
gctacatccccccctgta

SEQ ID NO: 21 - DNA, 20 bp, Papillomavirus sylvilagi
aagccacaagctttagggaa

SEQ ID NO: 22 - DNA, 19 bp, Papillomavirus sylvilagi
ccaaggaggatcgctatgc

SEQ ID NO: 23 - DNA, 22 bp, Papillomavirus sylvilagi
tgatttacgagacaagttttcc

SEQ ID NO: 24 - DNA, 18 bp, Papillomavirus sylvilagi
accgcctccactgctgca

SEQ ID NO: 25 - DNA, 20 bp, Papillomavirus sylvilagi
gcgacacacggaggaatttg

SEQ ID NO: 26 - DNA, 19 bp, Papillomavirus sylvilagi
gcctacccgtcccaaggtg

SEQ ID NO: 27 - DNA, 19 bp, Papillomavirus sylvilagi
cccgtatgcgcaaatgaca

SEQ ID NO: 28 - DNA, 21 bp, Papillomavirus sylvilagi
ggaatctgaggatccatatgc

SEQ ID NO: 29 - DNA, 19 bp, Papillomavirus sylvilagi
ctgagggaaaatcagaggc

SEQ ID NO: 30 - DNA, 21 bp, Papillomavirus sylvilagi
tgcaatacatacacaccatga

SEQ ID NO: 31 - DNA, 21 bp, Papillomavirus sylvilagi
catcccctccaacagcaaagg

SEQ ID NO: 32 - DNA, 22 bp, Papillomavirus sylvilagi
gtactgtatctgcacaatctgc

SEQ ID NO: 33 - DNA, 20 bp, Papillomavirus sylvilagi
ctgcacaatctgcatctgcc

SEQ ID NO: 34 - DNA, 21 bp, Papillomavirus sylvilagi
cttaccactgatgtaatggcc

SEQ ID NO: 35 - DNA, 18 bp, Papillomavirus sylvilagi
gcgatgcccctgcacagc